(19) United States
(12) Patent Application Publication
(10) Pub. No.: US 2026/0166118 A1
(43) Pub. Date: Jun. 18, 2026

(54) Extracellular MiRNA Motifs for Inflammation, Methods of Discovery and Uses Thereof

(71) Applicant: University of Maryland, Baltimore, Baltimore, MD (US)

(72) Inventors: Fu-Ming HU, Ellicott City, MD (US); Shiming Yang, Ellicott City, MD (US); Wei Chao, Ellicott City, MD (US); Boyang Ren, Baltimore, MD (US)

(73) Assignee: University of Maryland, Baltimore, Baltimore, MD (US)

(21) Appl. No.: 19/420,493

(22) Filed: Dec. 15, 2025

Related U.S. Application Data
(60) Provisional application No. 63/734,468, filed on Dec. 16, 2024.

(51) Int. Cl. A61K 38/00; C12N 15/113; C12Q 1/6883

(52) U.S. Cl. CPC A61K 38/00; C12N 15/113; C12Q 1/6883

(57) ABSTRACT

Computer-implemented and user-implemented methods for machine-learning guided discovery and identification of pro-inflammatory motifs in microRNAs and a non-transitory computer-readable medium tangibly storing processor-executable instructions to perform the method. Also provided is a set of pro-inflammatory motif nucleotide sequences identified by the method and a method for identifying pro-inflammatory extracellular-miRNAs in a biological sample using the motifs. In addition, an antagonist of a Toll-like receptor protein and a method for inhibiting miRNA-mediated inflammation in a subject by administering the antagonist.

### CROSS-REFERENCE TO RELATED APPLICATIONS

This non-provisional patent application claims benefit of priority under 35 U.S.C. § 119 (e) of provisional application U.S. Ser. No. 63/734,468, filed Dec. 16, 2024, the entirety of which is hereby incorporated by reference.

### STATEMENT REGARDING FEDERALLY SPONSORED RESEARCH

This invention was made with government support under grant GM140822 awarded by the National Institutes of Health. The government has certain rights in the invention.

### INCORPORATION BY REFERENCE OF SEQUENCE LISTING

The Sequence Listing XML file, entitled D8064SEQ.xml, was created on Dec. 12, 2025, and has a size of 85,000 bytes. This Sequence Listing is hereby incorporated by reference in its entirety.

### BACKGROUND OF THE INVENTION

### Field of the Invention

The present invention relates generally to the fields of biomarker identification and machine learning. More specifically, the present invention relates to the machine learning-guided discovery of extracellular miRNA (ex-miRNA) motifs as indicators of inflammation and uses thereof in designing therapeutic antagonists.

### Description of the Related Art

Trauma is a major cause of death for a wide span of ages worldwide (3,4). A recent study observed a rapid and dramatic increase in plasma single-stranded miRNA in animals and humans with trauma injury (1). Studies have uncovered the role of single-stranded extracellular miRNAs (ex-miRNAs) as damage-associated molecular patterns (DAMPs) to activate body's innate immune response via TLR7 and induce inflammation, in addition to their well-known gene regulatory functions via base-paring and silencing mechanism in the cell (5,6).

Importantly, not all ex-miRNAs cause inflammation. Hence, it is critical to identify those that are able to directly trigger immune responses in order to understand the pathophysiology of trauma-induced inflammation and develop potential prognostic biomarkers. Studies have shown nucleotide preferences, such as uridine dimers, in binding with TLR7 (2,7) and a more favorable role for uridines within the miRNA sequence in triggering an immune response (8).

Toll-like receptors (TLRs) are an important part of human innate immunity. There are at least 10 different types of Toll-like receptors. Studies have demonstrated the critical role of Toll-like receptors not only in host immune defense against invading pathogens but also in the pathogenesis of certain diseases such as autoimmune diseases, trauma, cardiac ischemic injury, and sepsis when such immune response becomes imbalanced and excessive.

Toll-like receptor 7 (TLR7) is an important innate immune sensor in humans for virus invasion. Upon invasion of virus into the cell, the intracellular TLR7 detects conserved single-stranded RNA (ssRNA) structure in the viral genome, followed by a signaling cascade leading to pro-inflammatory response in affected cell. Recent study shows that TLR7 can also be activated by endogenous ssRNA such as miRNAs (9). A significant elevation of plasma microRNA (miRNA) has been reported, the largest proportion of plasma ssRNA content, in both trauma and sepsis patients and exaggerated miRNA/TLR7-dependent inflammation responses. These inflammatory conditions, if left uncontrolled, would lead to severe immunopathology, in its worst scenario, cause death.

To date, there are 35 different TLR7 agonists that are either under or have completed clinical trials in the US, but just five TLR7 antagonists with active or completed status. Within these five antagonists, only one, IMO-3100, showed positive results in humans. Notably, there is lack of knowledge on the efficacy of TLR7 antagonists in the treatment of sepsis, trauma, and heart ischemic injury, where the pathogenic role of ex-miRNAs and TLR7 signaling in animal models has been found.

Thus, there remain unmet needs in the art for a motif-based classifier for miRNA-induced inflammation and organ injuries and therapeutic antagonists of TLR based on the pro-inflammatory motifs identified. Particularly, the art is deficient in an automated computational algorithm pipeline to identify nucleotide motifs correlated with the pro-inflammatory property of miRNAs and antagonists of TLR7 designed based on the identified motifs. The present invention fulfils this longstanding need and desire in the art.

### SUMMARY OF THE INVENTION

The present invention is directed to a computer-implemented method for identifying pro-inflammatory motifs in microRNAs (miRNA) where the method is performed by at least one computing device. In this method, a motif list is built and a motif existence list is constructed therefrom. An automated motif-finding algorithm is executed on the motif existence list.

The present invention is further directed to a set of pro-inflammatory motif nucleotide sequences identified by the computer-implemented method described herein.

The present invention is directed further to a method for identifying pro-inflammatory extracellular-miRNAs (ex-miRNA) in a biological sample. In the method the pro-inflammatory ex-miRNAs are captured via the set of pro-inflammatory motif nucleotide sequences described herein.

The present invention is directed further still to an antagonist of a Toll-like receptor (TLR) protein. The antagonist comprises an endogenous miRNA-derived nucleotide sequence.

The present invention is directed further still to a method for inhibiting miRNA-mediated inflammation in a subject. In this method, a therapeutically effective amount of the antagonist described herein is administered to the subject.

The present invention is directed further still to a non-transitory computer-readable medium containing processor-executable instructions, that when executed by the processor causes the computer to scan a training sequence of pro-inflammatory and non-inflammatory microRNAs (miRNA) for motifs with two to ten nucleotides, to create a motif existence list from the motifs consisting of two to ten nucleotides, to identify motifs from the motif existence list associated with inflammatory properties of the miRNA sequences, and to execute an automated motif-finding algorithm to identify the motifs most relevant to inflammatory responses.

The present invention is directed further still to a user-implemented method for machine-learning guided discovery of microRNA (miRNA) pro-inflammatory sequence motifs that directly predict a pro-inflammatory function. In this method, miRNA mimics are synthesized from a pro-inflammatory miRNA nucleotide sequence. Macrophage cells are cultured and are transfected with the miRNA mimics. A pro-inflammatory response induced by the miRNA mimics is quantified in the macrophage cells; where miRNA mimics are classified as pro-inflammatory or non-inflammatory based on the pro-inflammatory response. An automated motif finding algorithm tangibly stored in at least one computing device comprising a memory and a processor is applied, where the algorithm enables processor-executable instructions configured for: receiving as input the miRNA nucleotide sequences classified as pro-inflammatory or non-inflammatory, discovering and optimizing all theoretical k-mers that include wildcard positions, executing an automated motif finding process, outputting a classification table of a validation set of miRNAs, and calculating performance of a motif guided prediction.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 is a flowchart outlining the motif identification algorithm. The process begins with a list of miRNA sequences. It involves generating all possible sequence patterns, expanding the list to include patterns with wildcards, calculating pattern existence within each sequence, and applying an automated motif-finding algorithm (LASSO regression) to identify the most relevant motifs exclusively associated with pro-inflammatory miRNA activity.

FIG. 2 shows the correlation coefficients between nucleotides and outcome group.

FIGS. 3A-3C show the logistic regression results comparing four models using all nucleotides (AUCG), only A, only U, and combined A and U on the Training Set (FIG. 3A), on the Validation Set 1 (FIG. 3B) and on the Validation Set 2 (FIG. 3C).

FIG. 4 shows the hyperparameter tuning results for the LASSO model. The dash curve represents the number of nonzero coefficient motifs in the model, while the solid curve shows the corresponding F1 score for each model. The light grey dashed line indicates the decision point for the optimal LASSO-model, where it has the highest F1 score with the fewest motifs.

FIGS. 5A-5B shows the steps to reduce the original number of tested motifs to the 5 pro-inflammatory motifs (FIG. 5A) and the motif logo of the five identified pro-inflammatory illustrating the relative frequencies of the nucleotide at each position.

FIG. 6 shows the number of pro-inflammatory miRNAs captured by the five identified motifs and the combination of the five motifs.

FIGS. 7A-7B show the validation results of the septic mice dataset (FIG. 7A) and the trauma human dataset (FIG. 7B).

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., +5-10% of the recited value) that one of ordinary skill in the art would consider equivalent to the recited value (e.g., having the same function or result). In some instances, the term “about” may include numerical values that are rounded to the nearest significant figure.

As used herein, the ordinal adjectives “first” and “second” unless otherwise specified are used to describe a common object, merely indicate that different instances of like objects are being referred to, and are not intended to imply that the objects so described must be in a given sequence, either temporally, spatially, in ranking, or in any other manner.

As used herein, the conditional language, such as, among others, “can”, “might”, “may”, “e.g.”, “for example”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

As used herein, the term “subject” refers to a human or animal suffering from an inflammatory response and/or organ injury such as, but not limited to, sepsis and trauma.

As used herein, the terms “computational algorithm pipeline”, “automated machine-learning pipeline” are used interchangeably.

As used herein, the term computing device refers to a machine, such as a computer or other electronic device, that generally may include a processor, a memory, at least one input apparatus, a display structure or output apparatus, at least one network connecting means. The computing device may include at least one information storage/retrieval apparatus and/or means to store/retrieve information, such as, for example, a hard drive, a disk drive or a flash drive or memory stick, or other non-transitory computer readable media or non-transitory storage device, as is known in the art.

In one embodiment of the present invention, there is provided a computer-implemented method for identifying pro-inflammatory motifs in microRNAs (miRNA), the method performed by at least one computing device and comprising building a motif list; constructing a motif existence list therefrom; and executing an automated motif-finding algorithm on the motif existence list. In this embodiment, the miRNA may be an extracellular-miRNA (ex-miRNA).

In one aspect of this embodiment, the building step may comprise inputting a list of microRNA sequences containing both pro-inflammatory and non-inflammatory sequences, generating all possible sequence patterns and expanding the list to include patterns with wildcards.

In another aspect of this embodiment, the constructing step may comprise calculating pattern existence within each sequence to produce a motif existence list. In this aspect, the calculating step may comprise refining the pattern existence list to exclude 1) motifs appearing in both pro-inflammatory and non-inflammatory sequences and 2) motifs absent below a pre-determined frequency threshold from either pro-inflammatory and non-inflammatory sequences.

In yet another aspect of this embodiment, the executing step may comprises applying an automated motif-finding algorithm to identify motifs associated with pro-inflammatory miRNA activity and outputting a list of pro-inflammatory motifs.

In yet another aspect of this embodiment, the applying step may comprise tuning the automated motif-finding algorithm to remove redundant motifs whereby remaining motifs are key motifs linked to inflammation. Further to this aspect, the method comprises validating the key motifs as only pro-inflammatory motifs against inflammatory miRNAs. In both aspects 41 key motifs shown in Table 6 were validated as pro-inflammatory.

In another embodiment of the present invention, there is provided a set of pro-inflammatory motif nucleotide sequences identified by the computer-implemented method as described supra. In this embodiment, the set of pro-inflammatory motif nucleotide sequences may comprise UUC, U. . UU., .A . . . UU., G.UU. and UU . . . U.

In yet another embodiment of the present invention, there is provided a method for identifying pro-inflammatory extracellular-miRNAs (ex-miRNA) in a biological sample, comprising capturing the pro-inflammatory ex-miRNAs via the set of pro-inflammatory motif nucleotide sequences as described supra.

In this embodiment, the set of pro-inflammatory motif nucleotide sequences may be effective to capture the pro-inflammatory ex-miRNAs with a sensitivity and a specificity of 100%. In this embodiment, the set of pro-inflammatory motif nucleotide sequences may be effective to capture the pro-inflammatory ex-miRNAs with a sensitivity and a specificity of 100%. In addition, the biological sample may be from a subject with a traumatic injury or sepsis.

In yet another embodiment of the present invention, there is provided an antagonist of a Toll-like receptor (TLR) protein, comprising an endogenous miRNA-derived nucleotide sequence.

In this embodiment, the TLR protein may be TLR7. Also, in this embodiment, the endogenous miRNA may be Has-miR-146a-5p. In addition, the antagonist may have a nucleotide sequence of SEQ ID NO: 96, SEQ ID NO: 97 or SEQ ID NO: 98.

In yet another embodiment of the present invention, there is provided a method for inhibiting miRNA-mediated inflammation in a subject, comprising administering to the subject a therapeutically effective amount of the antagonist as described supra. In this embodiment, the miRNA-mediated inflammation in the subject may be from a trauma or sepsis.

In yet another embodiment of the present invention, there is provided a non-transitory computer-readable medium containing processor-executable instructions, that when executed by the processor causes the computer to scan a training sequence of pro-inflammatory and non-inflammatory microRNAs for motifs with two to ten nucleotides; create a motif existence list from the motifs consisting of two to ten nucleotides; identify motifs from the motif existence list associated with inflammatory properties of the miRNA sequences; and execute an automated motif-finding algorithm to identify the motifs most relevant to inflammatory responses.

In this embodiment, the processor-executable instruction to execute the automated motif-finding algorithm may cause the computer to refine the pattern existence list to exclude motifs appearing in both pro-inflammatory and non-inflammatory sequences and motifs absent below a pre-determined frequency threshold from of either pro-inflammatory and non-inflammatory sequences; and tune the automated motif-finding algorithm to remove redundant motifs whereby remaining motifs are key motifs linked to inflammation.

In yet another embodiment of the present invention, there is provided a user-implemented method for machine-learning guided discovery of microRNA pro-inflammatory sequence motifs that directly predict a pro-inflammatory function, comprising synthesizing miRNA mimics a from a pro-inflammatory miRNA nucleotide sequence; culturing macrophage cells; transfecting the macrophage cells with the miRNA mimics; quantifying a pro-inflammatory response induced by the miRNA mimics in the macrophage cells; said miRNA mimics classified as pro-inflammatory or non-inflammatory based on the pro-inflammatory response; applying an automated motif finding algorithm tangibly stored in at least one computing device comprising a memory and a processor, where the algorithm enabling processor-executable instructions is configured for: receiving as input the miRNA nucleotide sequences classified as pro-inflammatory or non-inflammatory; discovering and optimizing all theoretical k-mers that include wildcard positions; executing an automated motif finding process; outputting a classification table of a validation set of miRNAs; and calculating performance of a motif guided prediction.

In one aspect of this embodiment, the processor-executable instructions configured for discovering and optimizing all theoretical k-mers that include wildcard positions may comprise inputting the miRNA sequences; setting an inclusive k-mer range bounded by kmin and kmax; generating k-mer candidates; expanding motif candidates with wildcards; calculating motif presence and counts across the motif candidates; and assembling output tables comprising an existence matrix representing a presence or absence of the motif in the miRNA sequence and a count table of the number of each motif within the miRNA sequence.

In another aspect of this embodiment, the processor-executable instruction configured for enabling the automated motif finding process may comprise reading a training dataset and a testing dataset from a sequence workbook; loading the existence table and creating a feature table therefrom with the miRNA sequences as rows and the motifs as columns for training; and training a model via cross-validation on positive motifs from the training dataset to output a final motif list.

Provided herein is an automated machine-learning pipeline to identify nucleotide motifs correlated with the pro-inflammatory property of ex-miRNAs. The machine learning pipeline utilized L1 regularized logistic regression to automatically identify potential motif expressions that are exclusively present in the pro-inflammatory group and absent in the non-inflammatory group. Representative methods to determine the final motif listing include, but are not limited to, LASSO, elastic net, or other model with feature selection, such as, regularized regression model, “feature selection algorithm, sparsity-inducing machine learning model, including any model that drives coefficients to zero to select features.

Forty-one motifs exclusive to the pro-inflammatory, not the non-inflammatory miRNAs, were identified of which the set of five motifs.UUC, U . . . UU., .A . . . . UU., G.UU. and UU . . . U are the most inflammation related demonstrating high sensitivity and specificity. In combination, the five motifs accurately identified all pro-inflammatory miRNAs within the training group in the machine-learning pipeline. It is contemplated that this set of motifs may be used to identify proinflammatory ex-miRNAs from a large number of plasma candidates under various critical conditions in a subject, such as, but not limited to, trauma and sepsis.

Also provided are a new class of miRNA-derived TLR7 antagonists, named MD1, MD2, and MD3 (Table 8). These TLR7 antagonists were discovered based on the specific nucleotide motifs of miRNAs identified via the computational algorithm pipeline and that are responsible for miRNA-mediated pro-inflammatory property. Moreover, methods of reducing trauma induced inflammation in conditions such as, but not limited to, sepsis and organ injury.

The following examples are given for the purpose of illustrating various embodiments of the invention and are not meant to limit the present invention in any fashion.

### EXAMPLE 1

### Methods and Materials

### Datasets for Algorithm Development and Validation

Three datasets were used for machine learning algorithm development and validation. The first dataset, comprising 33 mouse ex-miRNAs from previously published research (2), was used for algorithm development. This dataset exhibited distinct and bifurcated pro-inflammatory cytokine production upon addition to cultured mouse bone marrow-derived macrophages, with 18 miRNAs showing dose-dependent production of MIP-2 and IL-6 (pro-inflammatory), while the other 15 did not (non-inflammatory) (Table 1).

TABLE 1
Trauma mouse miRNAs training set

| SEQ | | | |
|---|---|---|---|
| ID | miRNA | 5′-3′ Sequence | Inflammatory |
| 1 | hsa-miR-193b-3p | AACUGGCCCACAAAGUCCCGCU | Non- |
| | | | inflammatory |
| 2 | hsa-miR-345-3p | CCUGAACUAGGGGUCUGGAGAC | Non- |
| | | | inflammatory |
| 3 | hsa-miR-1947-5p | AGGACGAGCUAGCUGAGUGCUG | Non- |
| | | | inflammatory |
| 4 | hsa-miR-210-3p | CUGUGCGUGUGACAGCGGCUGA | Non- |
| | | | inflammatory |
| 5 | hsa-miR-22-3p | AAGCUGCCAGUUGAAGAACUGU | Non- |
| | | | inflammatory |
| 6 | hsa-miR-192-5p | CUGACCUAUGAAUUGACAGCC | Non- |
| | | | inflammatory |
| 7 | hsa-miR-374b-5p | AUAUAAUACAACCUGCUAAGUG | Non- |
| | | | inflammatory |
| 8 | hsa-miR-126a-3p | UCGUACCGUGAGUAAUAAUGCG | Non- |
| | | | inflammatory |
| 9 | hsa-miR-451a | AAACCGUUACCAUUACUGAGUU | Non- |
| | | | inflammatory |
| 10 | hsa-miR-144-5p | GGAUAUCAUCAUAUACUGUAAGU | Non- |
| | | | inflammatory |
| 11 | hsa-miR-150-5p | UCUCCCAACCCUUGUACCAGUG | Non- |
| | | | inflammatory |
| 12 | hsa-miR-1a-3p | UGGAAUGUAAAGAAGUAUGUAU | Non- |
| | | | inflammatory |
| 13 | hsa-miR-26a-5p | UUCAAGUAAUCCAGGAUAGGCU | Non- |
| | | | inflammatory |
| 14 | hsa-miR-499-5p | UUAAGACUUGCAGUGAUGUUU | Non- |
| | | | inflammatory |
| 15 | hsa-miR-144-3p | UACAGUAUAGAUGAUGUACU | Non- |
| | | | inflammatory |
| 16 | hsa-miR-208a-3p | AUAAGACGAGCAAAAAGCUUGU | Pro- |
| | | | inflammatory |
| 17 | hsa-miR-146a-5p | UGAGAACUGAAUUCCAUGGGUU | Pro- |
| | | | inflammatory |
| 18 | hsa-miR-802-5p | UCAGUAACAAAGAUUCAUCCUU | Pro- |
| | | | inflammatory |
| 19 | hsa-miR-145a-5p | GUCCAGUUUUCCCAGGAAUCCCU | Pro- |
| | | | inflammatory |
| 20 | hsa-miR-133a-3p | UUUGGUCCCCUUCAACCAGCUG | Pro- |
| | | | inflammatory |
| 21 | hsa-miR-26b-5p | UUCAAGUAAUUCAGGAUAGGU | Pro- |
| | | | inflammatory |
| 22 | hsa-miR-122-5p | UGGAGUGUGACAAUGGUGUUUG | Pro- |
| | | | inflammatory |
| 23 | hsa-miR-186-5p | CAAAGAAUUCUCCUUUUGGGCU | Pro- |
| | | | inflammatory |
| 24 | hsa-miR-25-3p | CAUUGCACUUGUCUCGGUCUGA | Pro- |
| | | | inflammatory |
| 25 | hsa-miR-382-5p | GAAGUUGUUCGUGGUGGAUUCG | Pro- |
| | | | inflammatory |
| 26 | hsa-miR-215-3p | UCUGUCAUUCUGUAGGCCAAU | Pro- |
| | | | inflammatory |
| 27 | hsa-miR-181d-5p | AACAUUCAUUGUUGUCGGUGGGU | Pro- |
| | | | inflammatory |
| 28 | hsa-miR-145a-3p | AUUCCUGGAAAUACUGUUCUUG | Pro- |
| | | | inflammatory |
| 29 | hsa-let-7b | UGAGGUAGUAGGUUGUGUGGUU | Pro- |
| | | | inflammatory |
| 30 | hsa-miR-34a-5p | UGGCAGUGUCUUAGCUGGUUGU | Pro- |
| | | | inflammatory |
| 31 | hsa-miR-7a-5p | UGGAAGACUAGUGAUUUUGUUGU | Pro- |
| | | | inflammatory |
| 32 | hsa-miR-142a-3p | UGUAGUGUUUCCUACUUUAUGGA | Pro- |
| | | | inflammatory |
| 33 | hsa-let-7j | UGAGGUAUUAGUUUGUGCUGUUAU | Pro- |
| | | | inflammatory |

Two datasets were selected for the validation process: Validation set 1 consisted of 20 mouse miRNAs (Table 2) upregulated in septic mice and was mutually exclusive with the training data set (Table 2) used for algorithm development. Among the 20 miRNAs, 13 were pro-inflammatory and 7 were non-inflammatory determined by in vitro MIP-2 secretion in cultured murine macrophages which were transfected with different dose of miRNAs.

TABLE 2
Septic mouse miRNAs validation set 1

| SEQ | | | |
|---|---|---|---|
| ID | miRNA | 5′-3′ Sequence | Inflammatory |
| 34 | mmu-miR-10b-3p | CAGAUUCGAUUCUAGGGGAAUA | Pro- |
| | | | inflammatory |
| 35 | mmu-miR-15b-5p | UAGCAGCACAUCAUGGUUUACA | Pro- |
| | | | inflammatory |
| 36 | mmu-miR-20a-5p | UAAAGUGCUUAUAGUGCAGGUAG | Pro- |
| | | | inflammatory |
| 37 | mmu-miR-221-3p | AGCUACAUUGUCUGCUGGGUUUC | Pro- |
| | | | inflammatory |
| 38 | mmu-miR-223-5p | CGUGUAUUUGACAAGCUGAGUUG | Pro- |
| | | | inflammatory |
| 39 | mmu-miR-10a-5p | UACCCUGUAGAUCCGAAUUUGUG | Pro- |
| | | | inflammatory |
| 40 | mmu-miR-28a-3p | CACUAGAUUGUGAGCUGCUGGA | Pro- |
| | | | inflammatory |
| 41 | mmu-miR-148a-3p | UCAGUGCACUACAGAACUUUGU | Pro- |
| | | | inflammatory |
| 42 | mmu-miR-200a-3p | UAACACUGUCUGGUAACGAUGU | Non- |
| | | | inflammatory |
| 43 | mmu-miR-206-3p | UGGAAUGUAAGGAAGUGUGUGG | Non- |
| | | | inflammatory |
| 44 | mmu-miR-26b-3p | CCUGUUCUCCAUUACUUGGCUC | Pro- |
| | | | inflammatory |
| 45 | mmu-miR-98-5p | UGAGGUAGUAAGUUGUAUUGUU | Pro- |
| | | | inflammatory |
| 46 | mmu-miR-147-3p | GUGUGCGGAAAUGCUUCUGCUA | Pro- |
| | | | inflammatory |
| 47 | mmu-miR-215-5p | AUGACCUAUGAUUUGACAGAC | Non- |
| | | | inflammatory |
| 48 | mmu-miR-377-3p | AUCACACAAAGGCAACUUUUGU | Pro- |
| | | | inflammatory |
| 49 | mmu-miR-16-5p | UAGCAGCACGUAAAUAUUGGCG | Non- |
| | | | inflammatory |
| 50 | mmu-miR-141-3p | UAACACUGUCUGGUAAAGAUGG | Non- |
| | | | inflammatory |
| 51 | mmu-miR-143-5p | GGUGCAGUGCUGCAUCUCUGG | Non- |
| | | | inflammatory |
| 52 | mmu-miR-194-5p | UGUAACAGCAACUCCAUGUGGA | Non- |
| | | | inflammatory |
| 53 | mmu-miR-652-3p | AAUGGCGCCACUAGGGUUGUG | Pro- |
| | | | inflammatory |

Since the differences in miRNA sequences and TLR7/8 exist between murine and humans, another dataset (Table 3) to extend the predicting algorithm to humans was introduced. Validation set 2 consisted of 38 most upregulated and high abundance ex-miRNAs found in trauma patients. The 38 miRNAs are also mutually exclusive with the training dataset. Among these miRNAs, 25 were pro-inflammatory and 13 were non-inflammatory determined by their pro-inflammatory ability in vitro.

TABLE 3
Trauma human miRNAs validation set 2

| SEQ | | | |
|---|---|---|---|
| ID | miRNA | 5′-3′ Sequence | Inflammatory |
| 54 | hsa-let-7c-5p | UGAGGUAGUAGGUUGUAUGGUU | Pro- |
| | | | inflammatory |
| 55 | hsa-let-7i-3p | CUGCGCAAGCUACUGCCUUGCU | Pro- |
| | | | inflammatory |
| 56 | hsa-miR-10a-3p | CAAAUUCGUAUCUAGGGGAAUA | Non- |
| | | | inflammatory |
| 57 | hsa-miR-125a-5p | UCCCUGAGACCCUUUAACCUGUG | Pro- |
| | | A | inflammatory |
| 58 | hsa-miR-132-3p | UAACAGUCUACAGCCAUGGUCG | Non- |
| | | | inflammatory |
| 59 | hsa-miR-136-3p | CAUCAUCGUCUCAAAUGAGUCU | Pro- |
| | | | inflammatory |
| 60 | hsa-miR-141-3p | UAACACUGUCUGGUAAAGAUGG | Non- |
| | | | inflammatory |
| 61 | hsa-miR-143-3p | UGAGAUGAAGCACUGUAGCUC | Non- |
| | | | inflammatory |
| 62 | hsa-miR-143-5p | GGUGCAGUGCUGCAUCUCUGGU | Non- |
| | | | inflammatory |
| 63 | hsa-miR-145-3p | GGAUUCCUGGAAAUACUGUUCU | Pro- |
| | | | inflammatory |
| 64 | hsa-miR-148a-3p | UCAGUGCACUACAGAACUUUGU | Pro- |
| | | | inflammatory |
| 65 | hsa-miR-152-3p | UCAGUGCAUGACAGAACUUGG | Pro- |
| | | | inflammatory |
| 66 | hsa-miR-15a-5p | UAGCAGCACAUAAUGGUUUGUG | Pro- |
| | | | inflammatory |
| 67 | hsa-miR-15b-5p | UAGCAGCACAUCAUGGUUUACA | Pro- |
| | | | inflammatory |
| 68 | hsa-miR-181a-2-3p | ACCACUGACCGUUGACUGUACC | Non- |
| | | | inflammatory |
| 69 | hsa-miR-181c-3p | AACCAUCGACCGUUGAGUGGAC | Non- |
| | | | inflammatory |
| 70 | hsa-miR-193a-5p | UGGGUCUUUGCGGGCGAGAUGA | Pro- |
| | | | inflammatory |
| 71 | hsa-miR-193b-3p | AACUGGCCCUCAAAGUCCCGCU | Pro- |
| | | | inflammatory |
| 72 | hsa-miR-194-5p | UGUAACAGCAACUCCAUGUGGA | Non- |
| | | | inflammatory |
| 73 | hsa-miR-195-5p | UAGCAGCACAGAAAUAUUGGC | Pro- |
| | | | inflammatory |
| 74 | hsa-miR-199a/b-3p | ACAGUAGUCUGCACAUUGGUUA | Pro- |
| | | | inflammatory |
| 75 | hsa-miR-199b-5p | CCCAGUGUUUAGACUAUCUGUUC | Pro- |
| | | | inflammatory |
| 76 | hsa-miR-200a-3p | UAACACUGUCUGGUAACGAUGU | Non- |
| | | | inflammatory |
| 77 | hsa-miR-214-3p | ACAGCAGGCACAGACAGGCAGU | Non- |
| | | | inflammatory |
| 78 | hsa-miR-224-5p | UCAAGUCACUAGUGGUUCCGUUU | Pro- |
| | | AG | inflammatory |
| 79 | hsa-miR-23a-3p | AUCACAUUGCCAGGGAUUUCC | Pro- |
| | | | inflammatory |
| 80 | hsa-miR-27b-5p | AGAGCUUAGCUGAUUGGUGAAC | Pro- |
| | | | inflammatory |
| 81 | hsa-miR-29a-3p | UAGCACCAUCUGAAAUCGGUUA | Pro- |
| | | | inflammatory |
| 82 | hsa-miR-29c-3p | UAGCACCAUUUGAAAUCGGUUA | Pro- |
| | | | inflammatory |
| 83 | hsa-miR-30d-3p | CUUUCAGUCAGAUGUUUGCUGC | Pro- |
| | | | inflammatory |
| 84 | hsa-miR-30e-3p | CUUUCAGUCGGAUGUUUACAGC | Pro- |
| | | | inflammatory |
| 85 | hsa-miR-30e-5p | UGUAAACAUCCUUGACUGGAAG | Non- |
| | | | inflammatory |
| 86 | hsa-miR-335-3p | UUUUUCAUUAUUGCUCCUGACC | Pro- |
| | | | inflammatory |
| 87 | hsa-miR-345-5p | GCUGACUCCUAGUCCAGGGCUC | Non- |
| | | | inflammatory |
| 88 | hsa-miR-361-5p | UUAUCAGAAUCUCCAGGGGUAC | Non- |
| | | | inflammatory |
| 89 | hsa-miR-365a/b-3p | UAAUGCCCCUAAAAAUCCUUAU | Pro- |
| | | | inflammatory |
| 90 | hsa-miR-493-5p | UUGUACAUGGUAGGCUUUCAUU | Pro- |
| | | | inflammatory |
| 91 | hsa-miR-98-5p | UGAGGUAGUAAGUUGUAUUGUU | Pro- |
| | | | inflammatory |

### Motif Finding Algorithm

Sequence motifs within miRNAs that preferentially trigger pro-inflammatory immune responses were identified. The occurrence of adenosine (A), uridine (U), cytidine (C), and guanosine (G) at each position within motifs ranging from 2 to 10 nucleotides (nt) in length (i.e., 2-mer-10 mer) was considered, generating all possible combinations for each motif length. To enhance the flexibility and comprehensiveness of the analysis, wildcards also were incorporated into the motif search. Wildcards represent positions where any of the four nucleotides (A, U, C, or G) can occur, allowing for a wider range of potential motif matches. The algorithm is shown in Table 4. For example, the motif “A.A” represents a 3-nucleotide motif where the middle position may be any nucleotide.

TABLE 4
Pseudo-code of the algorithm: Sequence Processing
and k-mer Generation Optimization Process

Algorithm A: Sequence Processing and k-mer Generation Optimization

| | Input: | |
|---|---|---|
| | □ | A list of miRNA sequences. |
| | □ | Motif length, k, an integer, can be ranging from 2 to the shortest |
| | | sequence length in the miRNA sequence list. |
| | Output: A set of motifs with wildcards. | |
| 1. | Convert each sequence to lowercase. | |
| 2. | Define a prefix ‘!’ and construct prefixed sequences. Then concatenate all | |
| | the prefixed sequences into a single long string. | |
| | Function: k-mer Generation Optimization Process | |
| | | Input: Motif length k (k-mer) |
| | | Output: A set of k-mers with wildcard substitutions. |
| | 3. | Extract all possible substrings of length k (k-mers) from the |

| concatenated sequence from step 2. | |
|---|---|
| 3.a | Consider the concatenated sequence as a single continuous |
| | string of length L. |
| 3.b | For each position i in the concatenated sequence, where 0 ≤ i ≤ |
| | L−k, extract a substring of length k starting from position i until all |
| | possible starting positions for a k-mer in the concatenated |
| | sequence have been considered, resulting in a total of L−k+1 k- |
| | mers. |
| 3.c | Filter out k-mers containing the prefix character and duplicated |

k-mers.

| | |
|---|---|
| 4. | Generate k-mers with wildcards. |

| 4.a | Initialize a variable, output_variable=set( ), to store k-mers with |
|---|---|
| | wildcard substitutions. |
| 4.b | For each k-mer remained after step 3.c: Generate k-mers with |
| | wildcards at 1 to k positions. Each k-mer can have multiple |
| | wildcards, with a maximum of k wildcards per sequence. For |
| | each subset of positions, replace one or more characters with a |
| | wildcard (.). |
| 4.c | Add each modified k-mer to output_variable. The use of Python's |

set structure ensures uniqueness and avoid redundant
calculation.

End Algorithm

To train a computer algorithm to distinguish motifs explicitly embedded in pro-inflammatory miRNAs, a machine-learning algorithm to automatically find critical motifs linked to inflammation was created. The proposed algorithm can efficiently and rapidly search through millions of short sequences to identify the critical motifs is ensured. This training set with binary categorization allows one to build the algorithm to find out the most probable motifs uniquely existing in the pro-inflammatory miRNAs.

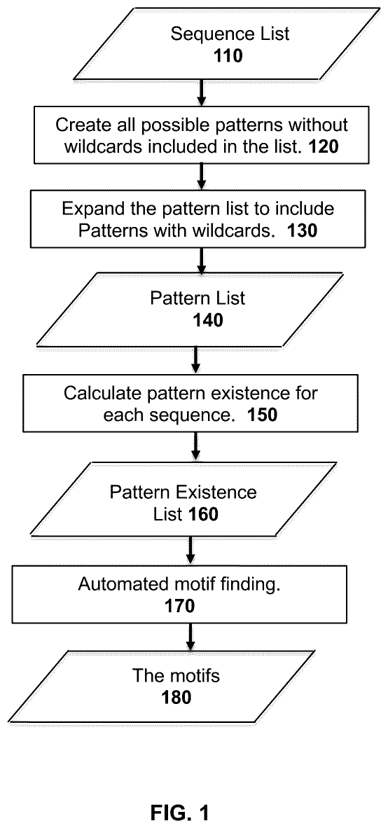

The algorithm consists of three main components: building a motif list, constructing a motif existence list, and executing an automated motif-finding algorithm. FIG. 1 shows the algorithm workflow. Generally, the sequence list in input at 110. All possible patterns with no wildcards are included in the list at 120 which is expanded the pattern list to include patterns with wildcards at 130. The pattern list is created at 140. The pattern existence is calculated for each sequence at 150 and the pattern existence list is created at 160. The next step is the automated motif finding at 170. The found motifs are outputted at 180.

Particularly, comprehensive scans were conducted for motifs ranging from two to ten nucleotides within the training miRNA sequences. This process yielded 513,599 motifs, including those with wildcards. Then, the presence of these motifs in each miRNA sequence was assessed to create a motif existence list. To further refine this list, the following were excluded 1) motifs that appear in both pro-inflammatory and non-inflammatory miRNA sequences or 2) if a motif is absent below a pre-determined frequency threshold, for example, absent in at least 25% of either group from the list. The goal is to identify optimal motifs strongly associated with the inflammatory properties of the miRNA sequences. Hence, if a motif is uncommon across the training set, it is considered less relevant for this purpose.

Following the reduction process, an automated motif-finding algorithm to pinpoint the most relevant motifs linked to inflammatory responses was developed. This approach utilized a penalized regression model called the “Least Absolute Shrinkage and Selection Operator” (LASSO) (7) to unravel critical motifs from the long motif existence list. The rationale for employing LASSO lies in its effectiveness as a feature selection tool in Data Science. In the training set, a 5-fold cross-validation was used and the hyperparameter, Lambda, that controls the strength of the L1 regularization term in the LASSO model was tuned. When the value of Lambda is set to 0, it indicates that the model has no penalty on the magnitude of the coefficients. As the value of Lambda increases, it introduces higher penalties on the magnitude of the coefficients, resulting in a sparser model. A higher Lambda value leads to the selection of fewer features in the model. By tuning the value of the Lambda in the LASSO model, the weights of redundant motifs are set to zero in the mathematical equation. In other words, a motif with a zero coefficient is considered non-essential, which is then removed in the regression model. As a result, the remaining motifs (motifs with nonzero weights) in the model are the key motifs linked to inflammation.

As each Lambda has a corresponding LASSO model, the question arises of how to determine the optimal Lambda, which means the optimal LASSO model with the best motif set. Thus, the prediction power of each LASSO model using the F1 score is evaluated, which is a performance evaluation metric for classification problems. The F1 score represents the harmonic mean of precision and recall. A higher F1 score indicates better predictive performance of the model. The optimal LASSO model was decided by the computer by having the highest train F1 score and the fewest motifs remaining in the model. In other words, the optimal motif list is the motifs used in the optimal LASSO model.

The algorithm's predictive performance was further validated by applying these identified motifs to the two validation sets, which consist of 20 mouse miRNAs (Validation set 1, Table 2) and 38 trauma human miRNA (Validation set 2, Table 3). Two performance metrics, sensitivity, and specificity, were used to evaluate the predictive performance of the two validation sets.

### EXAMPLE 2

Logistic Regression by the Single Nucleotide Features in miRNAs

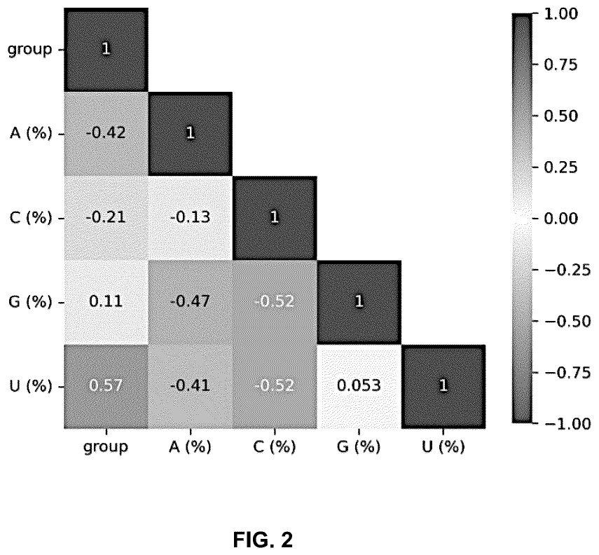

An introductory experiment to examine the abundance of nucleotides and their correlation with the outcome groups (pro-inflammatory vs. non-inflammatory) was conducted before the motif-finding process. FIG. 2 shows the correlation coefficient between each nucleotide and the outcome group. It shows that the abundance of U in the miRNA has a high positive correlation coefficient toward the outcome; the abundance of A has a high negative correlation toward the outcome, and there is no significant correlation for the abundance of C and G toward the outcome.

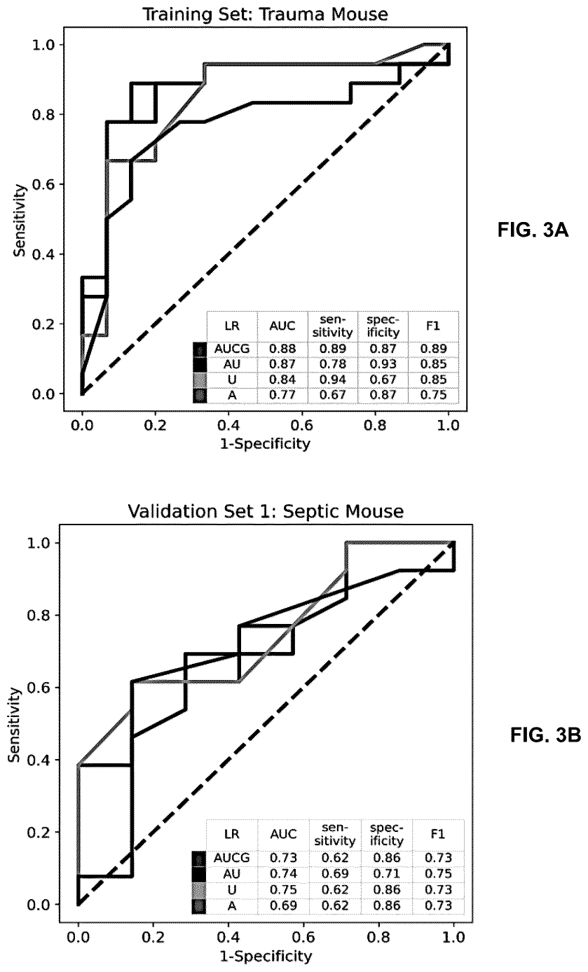

Based on the findings from FIG. 2, four logistic regression (LR) models using all nucleotides (AUCG), only A, only U, and A, and U combined was evaluated. The AUC curves in FIG. 3A show an AUC value of 0.89 for the AUCG model, an AUC value of 0.87 for the AU model, an AUC value of 0.84 for the U model, and an AUC value of 0.77 for the A model for the training set. Delong's test indicated no statistical significance in the performance of the four models. The tabular table in FIG. 3A lists the best classification result (sensitivity, specificity, and F1) each model could deliver. The threshold for each classification model was determined using the Youden index. FIGS. 3B-3C show the AUC curves of the Validation Set 1 and Validation Set 2 using the four trained LR. The classification results are listed in the tabular table in each figure.

miRNA Motif Selection by Lasso Regularization

Approximately half a million motifs with wildcards from the training set were identified. These motifs range from 2 to 10 nucleotides. Table 5 shows the number of motifs extracted for each nucleotide length from 2-mer to 10-mer.

TABLE 5
Number of motifs extracted for each length

| | Number of motifs | Number of motifs |
|---|---|---|
| Nucleotides | (without wildcards) | (with wildcards) |
| 2 | 16 | 25 |
| 3 | 64 | 125 |
| 4 | 214 | 583 |
| 5 | 422 | 2313 |
| 6 | 518 | 7681 |
| 7 | 514 | 22043 |
| 8 | 490 | 56512 |
| 9 | 460 | 132614 |
| 10 | 429 | 291703 |
| Total | 3127 | 513599 |

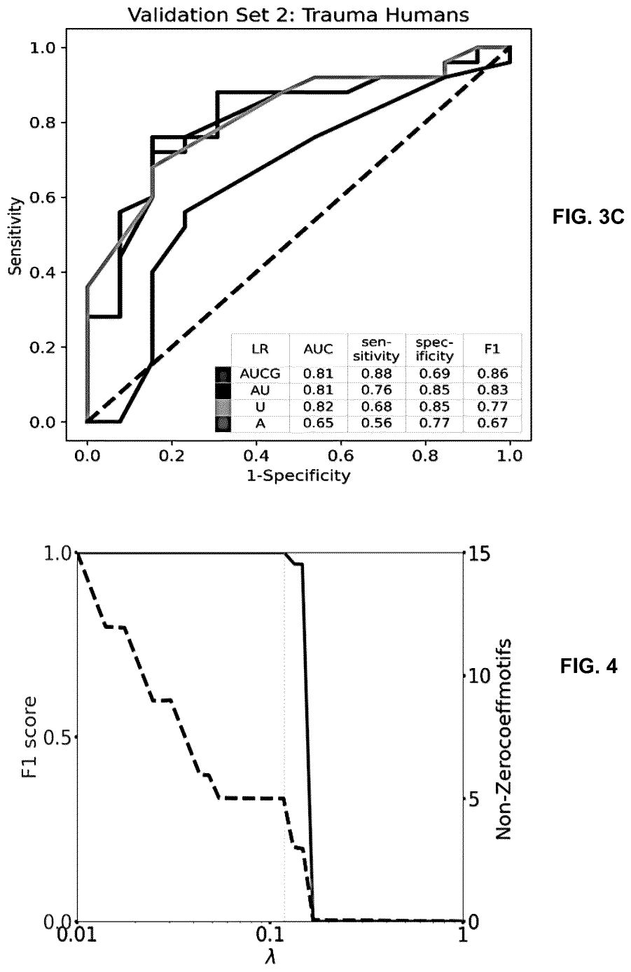

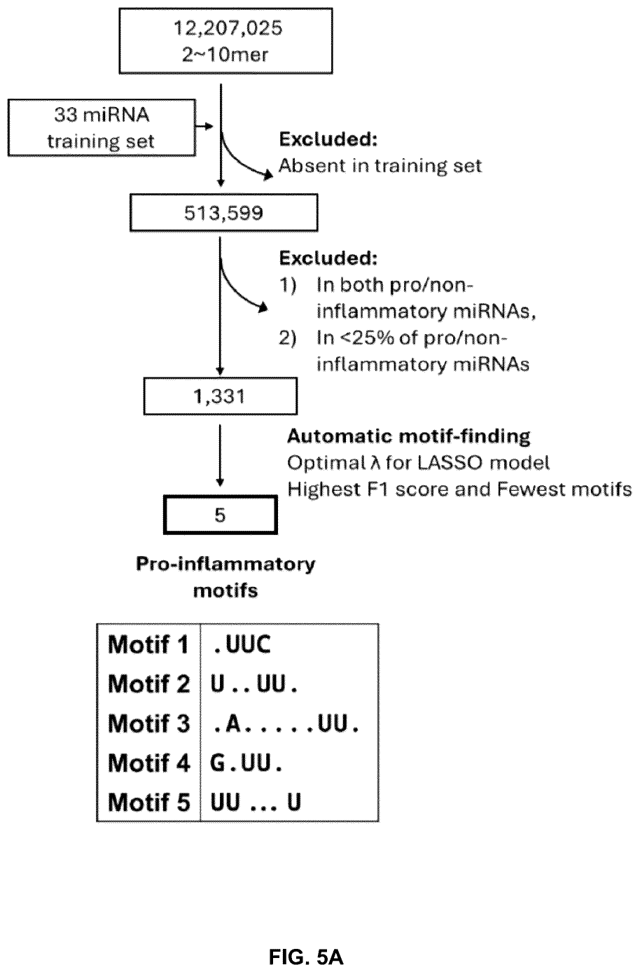

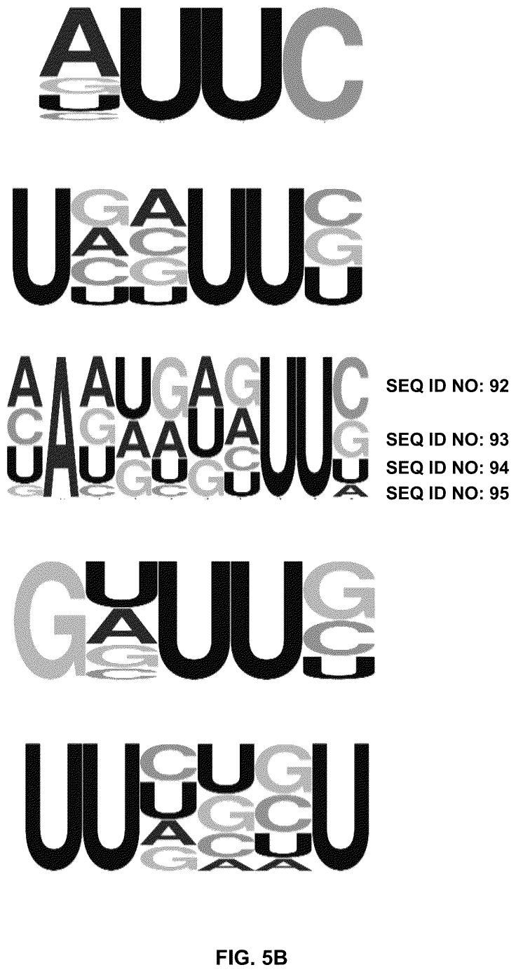

A machine learning-based automated algorithm is developed to discover the motifs for predicting the inflammatory properties of miRNAs from the half-a-million motifs pool. FIG. 4 shows hyperparameter tuning results on the LASSO model, varying the regulator Lambda from 0.01 to 1 and the corresponding F1 scores. As in Example 1, the optimal LASSO model was selected based on the highest training F1 score with the fewest motifs. In FIG. 4, the black dashed line indicated that at a Lambda value of 0.118, the highest F1 score of 1 was achieved, with only five motifs having nonzero coefficients in the LASSO model. This means that the algorithm processed approximately half a million motifs from the training dataset and identified the five motifs. UUC, U.. UU., .A . . . UU., G.UU. and UU . . . U as the most inflammation-related (FIG. 5A). The motif logos of the five motifs are shown in FIG. 5B. This frequency-based motif logos displays the frequency of nucleotides at each position. As shown in FIG. 6, each of these five motifs appears in 61.1% and 55.6% of the pro-inflammatory miRNA sequences, while the combination of the five motifs captures all 18 pro-inflammatory miRNAs. In other words, the automated motif-finding algorithm can automatically identify the motifs that, when applied together, can capture miRNAs with proinflammatory property with a sensitivity and a specificity of 100%.

Also, when using a relaxed hyperparameter Lambda value in the LASSO model, 41 motifs related to pro-inflammatory miRNA were found with a corresponding training F1 score of 1 (Table 6).

TABLE 6
Motifs

| # | Motif |
|---|---|
| 1 | .UUC |
| 2 | U..UU. |
| 3 | .A.....UU. |
| 4 | G.UU. |
| 5 | UU...U |
| 6 | A......UC |
| 7 | A......UUG |
| 8 | A...UUC |
| 9 | ..A..UU.. |
| 10 | A.U...U... |
| 11 | A.U...U..G |
| 12 | AG.UU |
| 13 | AUU.....U |
| 14 | AUUC |
| 15 | .AUUC |
| 16 | AUUC.. |
| 17 | .AUUC.. |
| 18 | .G.....C.U |
| 19 | G....U...U |
| 20 | G.UUG |
| 21 | GG.....U |
| 22 | GU....UU.. |
| 23 | GU.UU |
| 24 | U.....GG |
| 25 | U...C....U |
| 26 | U...UC |
| 27 | U...UC.... |
| 28 | U..G.U. |
| 29 | .U..U.C..U |
| 30 | U.GGU |
| 31 | U.GU...U.. |
| 32 | U.GUG.... |
| 33 | ....U.UC |
| 34 | .UC...C.. |
| 35 | UC..U |
| 36 | UGGU |
| 37 | UU...U.... |
| 38 | UU.G |
| 39 | .....UUC |
| 40 | ..UUC.... |
| 41 | UUU. |

Motif Validation in Separate miRNA Data Sets

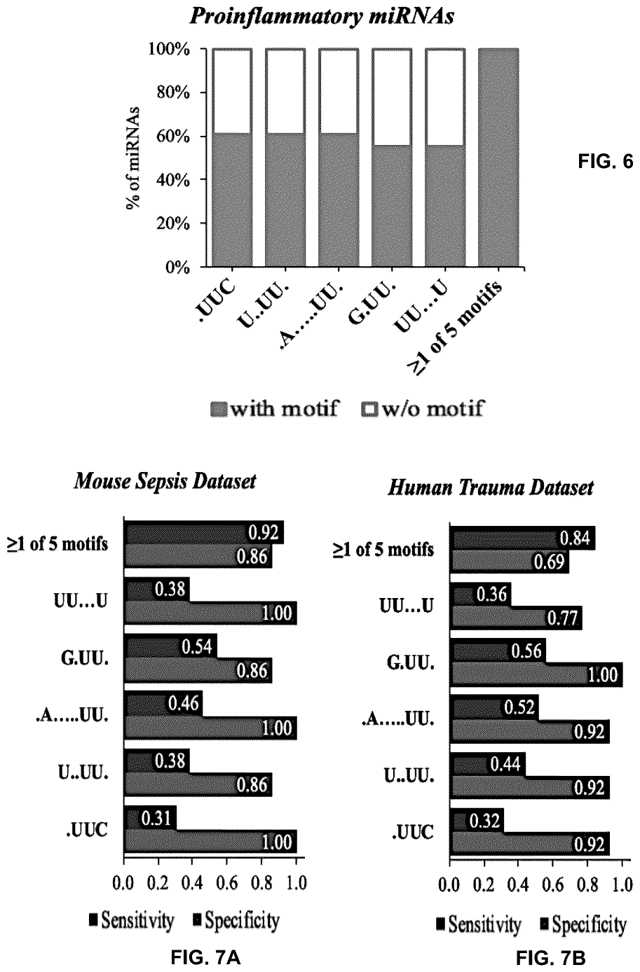

Two sets of different miRNAs that originated from mice and humans were used to validate the five motifs identified by the machine learning-based algorithm. Since the training set of miRNAs was from mice, the first validation set (Validation set 1) consisted of 20 mouse miRNAs upregulated in septic mice. This set was mutually exclusive with the training data set used for motif discovery. Out of these miRNAs, 13 were pro-inflammatory, and 7 were non-inflammatory. The combined five motifs can achieve an overall prediction sensitivity of 92% and specificity of 86%, while individual motifs provide much lower sensitivity than the combined (FIG. 7A). A second validation set (Validation set 2) was developed to extend the predictive algorithm to human miRNAs. The most upregulated ex-miRNAs with high abundance from 10 severe trauma patients were selected versus 10 healthy cohorts based on published plasma small RNase data (1). Validation set 2 consisted of a total of 38 mutually exclusive human miRNAs. Using the combined five motifs can achieve an overall prediction sensitivity of 84% and specificity of 69%, as individual motifs provide much lower sensitivity than the combined (FIG. 7B). From the results of these two validation datasets, it was observed that even though each motif has higher specificity than the combined five motifs, their corresponding sensitivity is much lower than the combined motifs. Table 7 shows the performance of motifs in capturing pro-inflammatory miRNAs.

TABLE 7
Performance of motifs

| Data set | Training set | | Test set 1 | | Test set 2 | |
|---|---|---|---|---|---|---|
| Origin | Mouse | | Mouse | | Human | |
| miRNA | 33 | | 20 | | 38 | |
| no. | | | | | | |
| | Sensi- | Speci- | Sensi- | Speci- | Sensi- | Speci- |
| | tivity | ficity | tivity | ficity | tivity | ficity |
| ≥1 motif | 100% | 100% | 92% | 86% | 84% | 69% |
| .UUC | 61% | 100% | 31% | 100% | 32% | 92% |
| U..UU. | 61% | 100% | 38% | 86% | 44% | 92% |
| .A.....UU. | 61% | 100% | 46% | 100% | 52% | 92% |
| SEQ ID | | | | | | |
| NO: 92 | | | | | | |
| G.UU. | 56% | 100% | 54% | 86% | 56% | 100% |
| UU...U | 56% | 100% | 38% | 100% | 36% | 77% |

### DISCUSSION

The introductory experiment provided fundamental insights into the association between specific miRNA nucleotides and inflammation. Analyzing the abundance of nucleotides within miRNA sequences provides a straightforward method for identifying potential biomarkers. Inflammatory miRNAs may have distinct nucleotide patterns, as shown in FIG. 2. Potential inflammatory biomarkers have stronger correlations with uracil (U) or adenine (A) nucleotides. Inflammatory miRNAs are identified by focusing on the nucleotide abundances without needing more complex structural analyses. Studies (2) have shown that miRNAs with a higher uracil abundance may have stronger binding relationships for TLR7/8, corresponding with the activation of inflammatory pathways. In other words, the abundance of specific nucleotides may influence miRNA's target binding ability and its interaction with proteins. Hence, by studying miRNAs based on nucleotide abundance, it is understood how these sequences regulate immune responses, particularly in inflammatory diseases.

The primary focus herein was to develop an automated motif-finding pipeline using machine learning and to identify miRNA motifs to predict the pro-inflammatory properties of miRNAs. Five key motifs predictive of inflammation with 100% sensitivity and specificity were identified in the discovery set and these motifs were validated in separate datasets from septic mice and trauma patients. However, the study used five identified motifs associated with inflammation in trauma mice miRNAs to predict inflammation in septic mice miRNAs and trauma human miRNAs. While this cross-species approach is valuable, it may also introduce challenges due to variations in miRNA sequences and their interactions with biological pathways. Future studies may focus on optimizing the algorithm's performance for more general applicability. Additionally, the current study identified motifs ranging from 2 to 10 nucleotides in length. Future studies could explore longer motifs to provide a more comprehensive understanding of the miRNA inflammatory process.

Analyzing the abundance of potential motifs found in a list of miRNAs is particularly challenging. For example, in this particular study, with only 33 training miRNAs, there were half a million motifs ranging from 2 to 10 nucleotides in length. Identifying motifs relevant to inflammation is the same as finding a needle in a haystack. To overcome this challenge, feature selection techniques from Data Science were utilized. Using LASSO, the most inflammatory-related motifs were identified from the tremendous amount of data in miRNA analysis.

Although the LASSO model was integrated for feature selection in the study, it may only capture some of the nuances of motif interactions. More advanced machine learning models like the Elastic Net or deep learning approaches may enhance motif discovery by capturing complex patterns and interactions. Also, the hyperparameters optimization process, such as the regulator Lambda in the LASSO model, significantly influences the model's performance. While these parameters are optimized based on the training dataset, further research is needed to explore the robustness of these findings across different hyperparameter values and to identify the optimal settings. Implementing advanced machine learning techniques could improve motif discovery by capturing more complex sequence pattern interactions and handling larger datasets more efficiently, potentially leading to more generalizable predictions.

The motifs found by the automated motif-finding algorithm are useful to identify pro-inflammatory miRNAs, which play crucial roles in trauma-induced inflammation or sepsis. The motifs discovered herein may serve as biomarkers for these conditions, assisting early diagnosis and personalized treatment strategies. Identifying and monitoring inflammation through the biomarkers could lead to significant advancements in patient care, particularly in managing chronic inflammatory conditions and trauma.

### EXAMPLE 3

miRNA Derived Toll-Like Receptor 7 (TLR7) Antagonists

A new class of miRNA-derived TLR7 antagonists, named MD1, MD2, and MD3 were designed and discovered based on the specific nucleotide motifs of miRNAs that are identified and are responsible for miRNA-mediated pro-inflammatory property. The MD family of TLR7 antagonists (Table 8) are the first of its kind that originated from miRNA with high potency of blocking TLR7. This was achieved by mutating a nucleotide that is essential for miRNA activation of TLR7 but without affecting the ligand-receptor binding. Moreover, the MD family of TLR7 antagonists are derived from endogenous miRNAs, rendering them minimally immunogenicity, and are amphipathic, enabling enhanced permeability into the intracellular compartment, where TLR7 is located.

TABLE 8
MD antagonist family

| | SEQ | | Motif |
|---|---|---|---|
| | ID | | X is |
| Name | NO: | Sequence | substitution |
| Original | 17 | UGAGAACUGAA | UU...U |
| Has-miR- | | UUCCAUGGGUU | |
| 146a-5p | | | |
| MD1 | 96 | AGAGAACAGAA | XX...X |
| | | AACCAAGGGAA | |
| MD2 | 97 | UGAGAACUGAA | XU...U |
| | | AUCCAUGGGUU | |
| MD3 | 98 | UGAGAACUGAA | UX....U |
| | | UACCAUGGGUU | |

In a cell-based test, MD1, the one most tested in this invention, selectively inhibits TLR7 activation by its ligand, but does not affect other TLR activation, such as TLR1, TLR2 and TLR4. The mechanistic study shows that MD1 acts through its binding to TLR7, as evidenced by two experiments: In the first experiment, pretreated macrophages were firstly pretreated with MD1, after that, culture medium so that cell-free MD1 were removed, under this scenario, MD1 pretreatment still inhibited TLR7 ligand-induced inflammatory response. This indicates MD1 functions not through sequestering the TLR7 ligand. In the second experiment, it was tested if MD1 can bind to highly related pro-inflammatory miRNA hsa-miR-146a-5p. By electrophoresis on double stranded nucleotide-sensitive gel, a size shift from 20 bp to 40 bp after incubation of MD1 with hsa-miR-146a-5p was not observed, besides, single-stranded RNA specific RNase T1 treatment fully digested the fragment, indicating no binding of MD1 to its potential ligand. Structurally similar derivatives MD2 and MD3 showed similar inhibition upon TLR7 activation.

### EXAMPLE 4

Machine-Learning Guided Pro-Inflammatory miRNA Motif Discovery: Algorithm

This computer-implemented method systematically identifies nucleotide motifs predicting miRNA pro-inflammatory properties using machine learning. The approach integrates in vitro functional validation in macrophages with computational exhaustive search and LASSO regression to discover sequence-specific motifs identifying pro-inflammatory miRNAs. This methodology provides a strategy for functional biomarker selection based on miRNA sequence, facilitating clinical translation without requiring extensive functional screening.

Synthesize miRNA Mimics

1. Enter miRBase (miRbase.org), type “miR-146a-5p” in the search box. miR-146a-5p is used as an example and positive pro-inflammatory control for the assay.

2. Click the human sequence name: hsa-miR-146a-5p and get the sequence as: UGAGAACUGAAUUCCAUGGGUU (SEQ ID NO: 17).

3. Add phosphorothioate (PS) bond modification to the ribonucleic backbone to enhance stability in the biological system (*: PS modification, r: RNA): rU*rG*r A*r G*r A*r A*r C*r U*r G*r A*r A*r U*r U*r C*r C*r A*r U*r G*r G*r G*r U*rU (SEQ ID NO: 17 from step 2).

4. Synthisize miRNA mimics from the vendor (RNA oligo synthesis service from Integrated DNA Technologies (IDT).

5. Reconstitute miRNA mimics with nuclease-free water to a stock concentration of 100 M.

### Reagent and Tools for Macrophage Culture

1. Culture medium

| | Final | Stock | | |
|---|---|---|---|---|
| Reagent | concentration | concentration | Volume | |
| DMEM (Gibco-11995-065) | NA | NA | 22.15 | mL |
| Fetal Bovine Serum | 10% | 100% | 2.5 | mL |
| Penicillin-Streptomycin | 100 U/mL | 10000 U/mL | 250 | uL |

(100x)

### Prewarm at 37° C.

2. Starvation medium. Total volume is 25 mL:

| | Final | Stock | | |
|---|---|---|---|---|
| Reagent | concentration | concentration | Volume | |
| DMEM | NA | NA | 24.5 | mL |
| 5% w/v BSA in PBS | 0.05% | 5% | 250 | uL |
| Penicillin-Streptomycin | 100 U/mL | 10000 U/mL | 250 | uL |

(100x)

### Prewarm at 37° C.

3. Other sterile materials are a flat-bottom 96-well plate (Falcon #353072, cell culture-treated).

### Computational Environment

Install python software and package:

1. Download and install the latest Python 3 version of Anaconda from anaconda.com/docs/getting-started/anaconda/install.

2. After installation, open Anaconda Navigator.

3. Install required python packages: Follow the steps in:
anaconda.com/docs/tools/anaconda-navigator/tutorials/manage-packages. Confirm the following packages are installed: pandas, numpy, xlsxwriter, scikit-learn, and matplotlib. If any are missing, install the missing packages through Anaconda Navigator as described in the webpage.

4. Verify the installation. Launch the latest version of Spyder through Anaconda Navigator. Run the following code snippets for each package. If there are no errors, the installation is complete.

>import pandas as pd
>print(pd.__version__)

### Macrophage Cell Culture: Part 1

1. Culture Raw 264.7 mouse macrophage cells in 10-cm cell culture dish until 80 confluency.

2. Scraper off the cells using a cell scrapper with soft rubber blade and transfer the cell suspension into a 50 ml conical tube.

3. Centrifuge at 300 g for 5 minutes at room temperature.

4. Remove supernatant and resuspend cells in 5 mL culture medium.

5. Count cell number and prepare cell suspension at the density of 3×10⁵/mL.

6. Dispense 100 uL cells per well into the non-edge wells of a 96-well plate as shown below.

7. To maintain consistent humidity and minimize edge effects, fill the outermost wells of the plate with PBS.

TABLE 9
Set-up of Raw 264.7 mouse macrophage cells in culture dish

| | 1 | 2 | 3 | 4 | 5 | 6 | 7 | 8 | 9 | 10 | 11 | 12 |
|---|---|---|---|---|---|---|---|---|---|---|---|---|
| A | PBS | PBS | PBS | PBS | PBS | PBS | PBS | PBS | PBS | PBS | PBS | PBS |
| B | PBS | Cells | Cells | Cells | Cells | Cells | Cells | Cells | Cells | Cells | Cells | PBS |
| C | PBS | Cells | Cells | Cells | Cells | Cells | Cells | Cells | Cells | Cells | Cells | PBS |
| D | PBS | Cells | Cells | Cells | Cells | Cells | Cells | Cells | Cells | Cells | Cells | PBS |
| E | PBS | Cells | Cells | Cells | Cells | Cells | Cells | Cells | Cells | Cells | Cells | PBS |
| F | PBS | Cells | Cells | Cells | Cells | Cells | Cells | Cells | Cells | Cells | Cells | PBS |
| G | PBS | Cells | Cells | Cells | Cells | Cells | Cells | Cells | Cells | Cells | Cells | PBS |
| H | PBS | PBS | PBS | PBS | PBS | PBS | PBS | PBS | PBS | PBS | PBS | PBS |

8. Incubate at 37° C. with 5% CO₂.

9. On the next day, cells are ready for transfection. Check the cell morphology under 10× objective microscope. Optimal conditions are 70% to 80% confluency where cells appearing bright and round.

Transfection of miRNA mimics: 20 hours (overnight) incubation: Part 2

TABLE 10
Final miRNA concentration (nM) in culture dish wells

| | 1 | 2 | 3 | 4 | 5 | 6 | 7 | 8 | 9 | 10 | 11 | 12 |
|---|---|---|---|---|---|---|---|---|---|---|---|---|
| A | | #1 | #2 | #3 | #4 | #5 | #6 | #7 | #8 | Pos | Neg | |
| B | | 500 | 500 | 500 | 500 | 500 | 500 | 500 | 500 | 500 | 0 | |
| C | | 500 | 500 | 500 | 500 | 500 | 500 | 500 | 500 | 500 | 0 | |
| D | | 50 | 50 | 50 | 50 | 50 | 50 | 50 | 50 | 50 | 0 | |
| E | | 50 | 50 | 50 | 50 | 50 | 50 | 50 | 50 | 50 | 0 | |
| F | | 5 | 5 | 5 | 5 | 5 | 5 | 5 | 5 | 5 | 0 | |
| G | | 5 | 5 | 5 | 5 | 5 | 5 | 5 | 5 | 5 | 0 | |
| H | | | | | | | | | | | | |

10. Cell Starvation.
a) Remove the previous medium carefully from the well, slowly add 100 uL prewarmed starvation medium.
b) Remove the starvation medium, add 90 uL starvation medium.
c) Keep culturing for 2 hours before transfection

11. miRNA working solution preparation.
a) Add 18 uL DMEM in a series of 3 tubes (tube A, B and C),
b) Add 2 uL 100 uM miRNA stock solution to tube A.
   i. For positive control, add 2 uL hsa-miR-146a-5p.
   ii. For negative control, add 2 uL DMEM.
c) Mix tube A with vortex, transfer 2 uL of tube A solution to tube B.
d) Mix tube B with vortex, transfer 2 uL of tube B solution to tube C.
e) Mix tube C with vortex.

12. Lipofectamine working solution preparation.
a) An equal amount of 0.15 □ L lipofectamine 3000 is used for transfection per well. To prepare lipofecamine for 60 wells of transfection, add 9 □ L lipofectamine 3000 into 29 □ L of DMEM, gently mix. Scale up and down accordingly.

13. Complex formation.
a) Dispense 12.5 □ L of miRNA working solution from tube A, B and C into separate new tubes.
b) Dispense 12.5 □ L lipofectamine working solution to each tube. Mix gently by flicking the tube.
c) Incubate the mixture for 15 minutes at room temperature.

14. Transfection.
d) Dispense 10 □ L miRNA-lipofectamine 3000 complex from tube A into the appropriate wells for a final transfection concentration of 500 nM. The complex are enough for duplicate wells.
e) Dispense 10 □ L complex from tube B to wells for 50 nM.
f) Dispense 10 □ L complex from tube C to wells for 5 nM.

15. Incubate at 37° C. with 5% CO₂

16. 18 hours later, transfer 60 □ L supernatant from each well into centrifuge tubes.

17. Centrifuge at 400 g for 10 minutes at 4° C.,

18. Transfer the supernatant to new tubes.

19. Samples can be used immediately or stored at −80° C. for the next step.

### Pro-Inflammatory Response Quantification and Classification (8 Hours): Part 3

20. Quantify the mouse pro-inflammatory chemokine CXCL2 following manufacturer's instructions.

21. Classification of the pro-inflammatory property. A miRNA is classified as pro-inflammatory if it meets both of the following criteria:
a) Dose-dependent increase: CXCL2 levels in culture medium show a continuous increase between 5 nM and 50 nM or between 50 nM and 500 nM.
b) Fold-change threshold: There is at least a 1.5-fold increase in CXCL2 production between any two adjacent tested miRNA concentrations. (50 nM vs 5 nM, or 500 nM vs 50 nM).

### Dose-Dependent Increase and Fold-Change Threshold

| | Dose-dependent | Fold-change | |
|---|---|---|---|
| miRNA | increase | threshold | Classification |
| miRNA A | Yes | Yes | Pro-inflammatory |
| miRNA B | No | Yes | Non-inflammatory |
| miRNA C | Yes | No | Non-inflammatory |
| miRNA D | No | No | Non-inflammatory |

22. Make a table of miRNA sequence and classification

Classification of miRNAs as Pro- or Non-Inflammatory

| miRNA | Sequence | Classification |
|---|---|---|
| miRNA A | . . . | Pro-inflammatory |
| miRNA B | . . . | Non-inflammatory |
| miRNA C | . . . | Non-inflammatory |
| miRNA D | . . . | Non-inflammatory |

### K-Mer Discovery and Optimization: Part 4

All theoretical k-mers that include wildcard positions are generated, where k is a value defined by the user. The value of k can range from 2 to N (any integer within the length of miRNA) and should be determined based on the available computational resources, as the number of candidate motifs increases exponentially with k. To manage memory usage and reduce processing time, only motifs that meet the specified criteria in the provided sequences are retained for further analysis. The timing is 10 seconds when using single-core computing and 3 seconds with parallel computing for calculating 2-10 mers.

23. Configure parameters.
a) Input file (seq_filename): Provide the Excel sheet containing the miRNA sequences. Ensure the file is accessible.
b) k-mer range (kmin, kmax): Set the inclusive bounds for the search and ensure that kmin is less than or equal to kmax.
c) Tolerance (tol): Define the group-frequency threshold, which states that a motif must occur in at least N_pos×tol miRNA sequences linked to a positive outcome. The value ranges from 0 to 1.
d) Parallel computing (is_parallel): Enable multiprocessing (TRUE) for faster processing or run in single-core mode (FALSE).
e) Result export (is_save_to_file), Output file (save_to_file): Decide whether to save outputs to an Excel file (TRUE) or not (FALSE). Specify the destination Excel filename when saving is enabled.

24. Generate k-mer candidates
a) Define the input sequence. Convert all sequences from the input Excel sheet to lowercase and concatenate them into one continuous string, adding a prefix to each sequence to signify the beginning of a new sequence.

>df[‘sequence’] = df[‘sequence’].str.lower( )
>prefix = “!”# or suffix
>prefix_seq = [prefix + sub for sub in seqs]
>prefix_seq = “.join(prefix_seq)

b) Slide a length-window across the sequence to enumerate all substrings.

| >def k_length_substrings(s, k): | |
|---|---|
| > | for i in range(len(s)-k+1): |
| > | yield s[i:i+k] |

> # Extract K length substrings
> res = list(k_length_substrings(prefix_seq, K))

c) Filter the substrings by removing any entry that contains the specified prefix token.

$$res\_remove \_prefix = [ s for s in res if prefix not in s ]$$

d) Deduplicate the remaining substrings to produce a unique k-mer list.

$$res\_unique = list ( set ( res\_remove \_prefix ) )$$

25. Expand Motif Candidates with Wildcards

Given a set of unique k-length strings, we generate all possible wildcard patterns by replacing any subset of positions with the dot character (.). We first define a list of positions where the wildcards can be applied, and then we create the full power set of these positions, which includes all possible combinations (masks). For each input motif candidate, we apply each mask to produce a pattern with dots inserted at the specified masked positions. Finally, we eliminate any duplicate motifs by storing them in a set.

| >wildcard_positions = list(range(K)) | |
|---|---|
| >output_set = set( ) | |
| >for key in res_unique: | |
| > | combinations = [[]] |
| > | for pos in wildcard_positions: |
| > | combinations +=[c + [pos]for c in combinations] |
| > | for comb in combinations: |
| > | this_str = list(key) |
| > | for index in comb: |
| > | this_str[index-1] = ‘.’ |
| > | output_set.add(“.join(this_str)) |

26. Calculate motif presence and counts across groups.
a) For each candidate motif, conduct a regular expression search across all sequences to create binary presence/absence profiles for each sequence.

$$exist\_array \_group = [ \text{} 1 if re . search ( pattern , string ) else 0 for string in seqs ]$$

b) Summarize the profiles separately for the positive and negative groups. Retain a k-mer only if it is frequent in the positive group (meeting the specified tolerance threshold) and completely absent in the negative group.
c) For all retained k-mers, calculate per-sequence counts of overlapping matches to capture abundance and store both the presence/absence profiles and the counts.

>exist_count_in_neg = sum(list(compress(exist_array_group, ind_neg)))
>exist_count_in_pos = sum(list(compress(exist_array_group, ind_pos)))
>if (exist_count_in_pos >= cutoff_count_pos and not

exist_count_in_neg) or (exist_count_in_neg >= cutoff_count_neg and not
exist_count_in_pos):

| | |
|---|---|
| > | count_array_group = [my_subfunc.my_overlappingcount(pattern, |

string) for string in seqs]

| | |
|---|---|
| > | count_list. append(count_array_group) |
| > | kmer_pool.append(pattern) |
| > | occurrence_list.append(exist_array_group) |

d) Assemble output tables.
   i. Construct an existence matrix, where rows represent motifs and columns indicate whether each motif exists in miRNA sequences.
   ii. Create a count table, where rows are motifs and columns show the abundance count of each motif within the miRNA sequences.
e) Save the output tables to the specified Excel file if the save option is enabled.

>if is_save_to_file:

| | |
|---|---|
| > | writer = pd.ExcelWriter(save_to_file, engine=‘xlsxwriter’) |
| > | df_kmer.to_excel(writer, sheet_name=‘existence’) |
| > | df_count.to_excel(writer, sheet_name=‘count’) |
| > | writer. close( |

The process can be run using parallel computing or not. If you decide to run the process without parallel computing, the script evaluates candidates sequentially across all k values. On the other hand, if you opt for parallel computing, it evaluates candidate k-mers concurrently using a worker pool. After processing, the script will merge the returned k-mers, presence/absence profiles, and the count vectors into the same two tables and provide the option to export them as well. Using parallel computing is beneficial when calculating larger k-mers, as it may significantly speed up the processing time.

### Perform the Automated Motif Finding Algorithm: Part 5

Timing is 1 second, the duration may vary depending on the number of LASSO modules being trained.

27. Prepare training and testing data for machine learning
a) Read the training and testing dataset from the sequence workbook, converting all sequences to lowercase for consistency.
b) Load the motif workbook (the existence sheet from the previous step) and remove duplicate rows, so each motif has a single, unique presence/absence profile.
c) Transpose the motif-by-sequence matrix to create a feature table, with sequences as rows and motifs as columns, for training purposes.
d) Scale features using min-max normalization, fit on the training set only, and apply the same transformation to all downstream data.
e) Apply the previous steps for the testing sequences and labels for subsequent prediction and evaluation.

28. Determine the final motif list using LASSO

A Lasso model is trained using five-fold cross-validation on the motif panel derived from the positive training group. The regularization strength by scanning a logarithmic grid ranging from 0.01 to 1, using 42 values was explored. For each value, the training F1 score was recorded, the number of non-zero coefficients, and the corresponding list of selected motifs (motifs with non-zero coefficients). The final model was determined by ranking according to the highest training F1 score, resolving ties first by preferring models with fewer selected motifs, and subsequently by choosing the model with the larger regularization value. The outputs include the fitted model, the selected motif coefficients, performance metrics across the grid, and a table that links each alpha value to its corresponding set of selected motifs in the Excel file. In addition, the code outputs the final motif list and visualizes the LASSO hyperparameter-tuning results in the Spyder IDE.

>nsample = 42
>alphas = np.logspace(−2, 0, nsample)
>lassocv = LassoCV(cv=5, max_iter=10000)
>exp1_df_final, exp1_coefs, exp1_df_perfmetrics,

exp1_df_motif_alpha_list = my_subfunc.my_LASSO(X_train_pos_motif, y_train,
df_seq_test, y_test, alphas, lassocv)

>results = {‘alphas’: alphas, ‘train_f1’: exp1_df_perfmetrics[‘train_f1’],

‘N_nonzeros’: exp1_df_perfmetrics[‘N_nonzeros’]}

>df_results = pd.DataFrame(results)
>df_results.sort_values(by=[‘train_f1’,‘N_nonzeros’,‘alphas’],

ascending=[False, True, False], inplace = True, ignore_index=False)

># get the final motif list
>final_decision_idx = df_results. index[0]
>s = exp1_df_motif_alpha_list[‘ListIndex’]
>matching_idx = s.index[s.eq(final_decision_idx)]
>final_decision_motifList = exp1_df_motif_alpha_list.loc[matching_idx,

‘motifs’]

>final_decision_alpha = df_results[‘alphas’][final_decision_idx]
>print(“The final motif list:”, final_decision_motifList.tolist( ))
29. Save the outputs to an Excel sheet

By enabling the save option, we write the fitting results from LASSO to an Excel file with separate sheets for future reference or in-depth analysis of the individual machine learning models

>if is_save_to_file:

| | |
|---|---|
| > | writer = pd.ExcelWriter(save_to_file, engine=‘xlsxwriter’) |
| > | exp1_df_motif_alpha_list.to_excel(writer, sheet_name=‘exp1_motif’) |
| > | exp1_df_perfmetrics.to_excel(writer, sheet_name=‘exp1_perfmetric’) |
| > | writer.close( ) |

Performance Estimation for Motif-Guided Prediction of Pro-Inflammatory miRNA: Part 6

30. Select a validation set of miRNAs set that are non-redundant from the training set used for motif discovery.

31. Obtain the experimental classification based on parts 1-3.

32. Obtain the motif-guide prediction based on part 5.

33. Make a classification table for the validation set of miRNAs: (Pro: pro-inflammatory, Non: non-inflammatory).

TABLE 11
miRNA validation set classification table

| | Experimental | Motif-guided | |
|---|---|---|---|
| | classification | prediction | Results |
| miRNA A | Pro | Pro | True positive (TP) |
| miRNA B | Pro | Non | False negative (FN) |
| miRNA C | Non | Pro | False positive (FP) |
| miRNA D | Non | Non | True negative (TN) |

34. Calculate performance of motif guided prediction:

$$Sensitivity = TP / ( TP + FN ) \%$$

$$Sensitivity = TN / ( FP + TN ) \%$$

The following references are cited herein.
1. Suen, A. O., et al. Extracellular RNA Sensing Mediates Inflammation and Organ Injury in a Murine Model of Polytrauma. J Immunol 210, 1990-2000 (2023).
2. Wang, S., et al. Role of extracellular microRNA-146a-5p in host innate immunity and bacterial sepsis. iScience. 24, 103441 (2021).
3. Rossiter, N. D. Trauma-the forgotten pandemic?. Int Orthop 46, 3-11 (2022).
4. (CDC), C.f.D.C.a.P. 10 Leading Causes of Death, United States. in 2022 (cdc.gov, USA, 2022).
5. Williams et al., Emerging Role of Extracellular RNA in Innate Immunity, Sepsis, and Trauma, Shock, 59 (2): 190-199, Epub Nov. 3, 2022.
6. Bartel, D. P. MicroRNAs: genomics, biogenesis, mechanism, and function. Cell 116, 281-297 (2004).
7. Zhang et al., Structural Analyses of Toll-like Receptor 7 Reveal Detailed RNA Sequence Specificity and Recognition Mechanism of Agonistic Ligands, Cell Rep., 25 (12): 3371-3381.e5, Dec. 18, 2018
8. Lehmann, S. M., et al. An unconventional role for miRNA: let-7 activates Toll-like receptor 7 and causes neurodegeneration. Nat Neurosci 15, 827-835 (2012).
9. Tibshirani, R., Regression Shrinkage and Selection via The Lasso: A Retrospective, Journal of the Royal Statistical Society Series B: Statistical Methodology 73, 273-282 (2011).

What is claimed is:

1. A computer-implemented method for identifying pro-inflammatory motifs in microRNAs (miRNA), the method performed by at least one computing device and comprising:
building a motif list;
constructing a motif existence list; and
executing an automated motif-finding algorithm.

2. The computer-implemented method of claim 1, wherein the building step comprises:
inputting a list of microRNA sequences containing both pro-inflammatory and non-inflammatory sequences;
generating all possible sequence patterns; and
expanding the list to include patterns with wildcards.

3. The computer-implemented method of claim 1, wherein the constructing step comprises:
calculating pattern existence within each sequence to produce a motif existence list.

4. The computer-implemented method of claim 3, wherein the calculating step comprises:
refining the pattern existence list to exclude:
motifs appearing in both pro-inflammatory and non-inflammatory sequences; and
motifs absent below a pre-determined frequency threshold from either pro-inflammatory and non-inflammatory sequences.

5. The computer-implemented method of claim 1, wherein the executing step comprises:
applying an automated motif-finding algorithm to identify motifs associated with pro-inflammatory miRNA activity; and
outputting a list of pro-inflammatory motifs.

6. The computer-implemented method of claim 1, wherein the applying step comprises:
tuning the automated motif-finding algorithm to remove redundant motifs whereby remaining motifs are key motifs linked to inflammation.

7. The computer-implemented method of claim 6, further comprising:
validating the key motifs as only pro-inflammatory motifs against inflammatory miRNAs.

8. The computer-implemented method of claim 7, wherein 41 key motifs shown in Table 6 were validated as pro-inflammatory.

9. The computer-implemented method of claim 1, wherein the miRNA is an extracellular-miRNA.

10. A set of pro-inflammatory motif nucleotide sequences identified by the computer-implemented method of claim 1.

11. The set of pro-inflammatory motif nucleotide sequences of claim 10, comprising UUC, U..UU., .A . . . UU., G.UU. and UU . . . U.

12. A method for identifying pro-inflammatory extracellular-miRNAs in a biological sample, comprising:
capturing the pro-inflammatory extracellular-miRNAs via the set of pro-inflammatory motif nucleotide sequences of claim 10.

13. The method of claim 12, wherein the set of pro-inflammatory motif nucleotide sequences are effective to capture the pro-inflammatory extracellular-miRNAs with a sensitivity and a specificity of 100%.

14. The method of claim 12, wherein the biological sample is from a subject with a traumatic injury or sepsis.

15. An antagonist of a Toll-like receptor protein, comprising:
an endogenous miRNA-derived nucleotide sequence.

16. The antagonist of claim 15, wherein the Toll-like receptor protein is TLR7.

17. The antagonist of claim 15, wherein the endogenous miRNA is Has-miR-146a-5p.

18. The antagonist of claim 15, consisting of the nucleotide sequence of SEQ ID NO: 96, SEQ ID NO: 97 or SEQ ID NO: 98.

19. A method for inhibiting miRNA-mediated inflammation in a subject, comprising:
administering to the subject a therapeutically effective amount of the antagonist of claim 15.

20. The method of claim 19, wherein the miRNA-mediated inflammation in the subject is from a trauma or sepsis.

21. A non-transitory computer-readable medium containing processor-executable instructions, that when executed by the processor causes the computer to:
scan a training sequence of pro-inflammatory and non-inflammatory microRNAs (miRNA) for motifs with two to ten nucleotides;
create a motif existence list from the motifs consisting of two to ten nucleotides;
identify motifs from the motif existence list associated with inflammatory properties of the miRNA sequences; and
execute an automated motif-finding algorithm to identify the motifs most relevant to inflammatory responses.

22. The non-transitory computer-readable medium of claim 21, wherein the processor-executable instruction to execute the automated motif-finding algorithm causes the computer to:
refine the pattern existence list to exclude motifs appearing in both pro-inflammatory and non-inflammatory sequences and motifs absent below a pre-determined frequency threshold from of either pro-inflammatory and non-inflammatory sequences; and
tune the automated motif-finding algorithm to remove redundant motifs whereby remaining motifs are key motifs linked to inflammation.

23. A user-implemented method for machine-learning guided discovery of microRNA pro-inflammatory sequence motifs that directly predict a pro-inflammatory function, comprising:
synthesizing microRNA mimics from a pro-inflammatory microRNA nucleotide sequence;
culturing macrophage cells;
transfecting the macrophage cells with the microRNA mimics;
quantifying a pro-inflammatory response induced by the microRNA mimics in the macrophage cells; said microRNA mimics classified as pro-inflammatory or non-inflammatory based on the pro-inflammatory response;
applying an automated motif finding algorithm tangibly stored in at least one computing device comprising a memory and a processor, said algorithm enabling processor-executable instructions configured for:
receiving as input the microRNA nucleotide sequences classified as pro-inflammatory or non-inflammatory;
discovering and optimizing all theoretical k-mers that include wildcard positions;
executing an automated motif finding process;
outputting a classification table of a validation set of microRNAs; and
calculating performance of a motif guided prediction.

24. The user-implemented method of claim 23, wherein the processor-executable instructions configured for discovering and optimizing all theoretical k-mers that include wildcard positions, comprises:
inputting the microRNA sequences;
setting an inclusive k-mer range bounded by kmin and kmax;
generating k-mer candidates;
expanding motif candidates with wildcards;
calculating motif presence and counts across the motif candidates; and
assembling output tables comprising an existence matrix representing a presence or absence of the motif in the microRNA sequence and a count table of the number of each motif within the microRNA sequence.

25. The user-implemented method of claim 23, wherein the processor-executable instruction configured for enabling the automated motif finding process, comprises:
reading a training dataset and a testing dataset from a sequence workbook;
loading the existence table and creating a feature table therefrom with the miRNA sequences as rows and the motifs as columns for training; and
training a model via cross-validation on positive motifs from the training dataset to output a final motif list.


SEQUENCE LISTING (98 sequences)

SEQ ID NO: 1 - RNA, 22 nt, synthetic construct
  source 1..22: mol_type = other RNA
aactggcccacaaagtcccgct

SEQ ID NO: 2 - RNA, 22 nt, synthetic construct
  source 1..22: mol_type = other RNA
cctgaactaggggtctggagac

SEQ ID NO: 3 - RNA, 22 nt, synthetic construct
  source 1..22: mol_type = other RNA
aggacgagctagctgagtgctg

SEQ ID NO: 4 - RNA, 22 nt, synthetic construct
  source 1..22: mol_type = other RNA
ctgtgcgtgtgacagcggctga

SEQ ID NO: 5 - RNA, 22 nt, synthetic construct
  source 1..22: mol_type = other RNA
aagctgccagttgaagaactgt

SEQ ID NO: 6 - RNA, 21 nt, synthetic construct
  source 1..21: mol_type = other RNA
ctgacctatgaattgacagcc

SEQ ID NO: 7 - RNA, 22 nt, synthetic construct
  source 1..22: mol_type = other RNA
atataatacaacctgctaagtg

SEQ ID NO: 8 - RNA, 22 nt, synthetic construct
  source 1..22: mol_type = other RNA
tcgtaccgtgagtaataatgcg

SEQ ID NO: 9 - RNA, 22 nt, synthetic construct
  source 1..22: mol_type = other RNA
aaaccgttaccattactgagtt

SEQ ID NO: 10 - RNA, 23 nt, synthetic construct
  source 1..23: mol_type = other RNA
ggatatcatcatatactgtaagt

SEQ ID NO: 11 - RNA, 22 nt, synthetic construct
  source 1..22: mol_type = other RNA
tctcccaacccttgtaccagtg

SEQ ID NO: 12 - RNA, 22 nt, synthetic construct
  source 1..22: mol_type = other RNA
tggaatgtaaagaagtatgtat

SEQ ID NO: 13 - RNA, 22 nt, synthetic construct
  source 1..22: mol_type = other RNA
ttcaagtaatccaggataggct

SEQ ID NO: 14 - RNA, 21 nt, synthetic construct
  source 1..21: mol_type = other RNA
ttaagacttgcagtgatgttt

SEQ ID NO: 15 - RNA, 20 nt, synthetic construct
  source 1..20: mol_type = other RNA
tacagtatagatgatgtact

SEQ ID NO: 16 - RNA, 22 nt, synthetic construct
  source 1..22: mol_type = other RNA
ataagacgagcaaaaagcttgt

SEQ ID NO: 17 - RNA, 22 nt, synthetic construct
  source 1..22: mol_type = other RNA
tgagaactgaattccatgggtt

SEQ ID NO: 18 - RNA, 22 nt, synthetic construct
  source 1..22: mol_type = other RNA
tcagtaacaaagattcatcctt

SEQ ID NO: 19 - RNA, 23 nt, synthetic construct
  source 1..23: mol_type = other RNA
gtccagttttcccaggaatccct

SEQ ID NO: 20 - RNA, 22 nt, synthetic construct
  source 1..22: mol_type = other RNA
tttggtccccttcaaccagctg

SEQ ID NO: 21 - RNA, 21 nt, synthetic construct
  source 1..21: mol_type = other RNA
ttcaagtaattcaggataggt

SEQ ID NO: 22 - RNA, 22 nt, synthetic construct
  source 1..22: mol_type = other RNA
tggagtgtgacaatggtgtttg

SEQ ID NO: 23 - RNA, 22 nt, synthetic construct
  source 1..22: mol_type = other RNA
caaagaattctccttttgggct

SEQ ID NO: 24 - RNA, 22 nt, synthetic construct
  source 1..22: mol_type = other RNA
cattgcacttgtctcggtctga

SEQ ID NO: 25 - RNA, 22 nt, synthetic construct
  source 1..22: mol_type = other RNA
gaagttgttcgtggtggattcg

SEQ ID NO: 26 - RNA, 21 nt, synthetic construct
  source 1..21: mol_type = other RNA
tctgtcattctgtaggccaat

SEQ ID NO: 27 - RNA, 23 nt, synthetic construct
  source 1..23: mol_type = other RNA
aacattcattgttgtcggtgggt

SEQ ID NO: 28 - RNA, 22 nt, synthetic construct
  source 1..22: mol_type = other RNA
attcctggaaatactgttcttg

SEQ ID NO: 29 - RNA, 22 nt, synthetic construct
  source 1..22: mol_type = other RNA
tgaggtagtaggttgtgtggtt

SEQ ID NO: 30 - RNA, 22 nt, synthetic construct
  source 1..22: mol_type = other RNA
tggcagtgtcttagctggttgt

SEQ ID NO: 31 - RNA, 23 nt, synthetic construct
  source 1..23: mol_type = other RNA
tggaagactagtgattttgttgt

SEQ ID NO: 32 - RNA, 23 nt, synthetic construct
  source 1..23: mol_type = other RNA
tgtagtgtttcctactttatgga

SEQ ID NO: 33 - RNA, 24 nt, synthetic construct
  source 1..24: mol_type = other RNA
tgaggtattagtttgtgctgttat

SEQ ID NO: 34 - RNA, 22 nt, synthetic construct
  source 1..22: mol_type = other RNA
cagattcgattctaggggaata

SEQ ID NO: 35 - RNA, 22 nt, synthetic construct
  source 1..22: mol_type = other RNA
tagcagcacatcatggtttaca

SEQ ID NO: 36 - RNA, 23 nt, synthetic construct
  source 1..23: mol_type = other RNA
taaagtgcttatagtgcaggtag

SEQ ID NO: 37 - RNA, 23 nt, synthetic construct
  source 1..23: mol_type = other RNA
agctacattgtctgctgggtttc

SEQ ID NO: 38 - RNA, 23 nt, synthetic construct
  source 1..23: mol_type = other RNA
cgtgtatttgacaagctgagttg

SEQ ID NO: 39 - RNA, 23 nt, synthetic construct
  source 1..23: mol_type = other RNA
taccctgtagatccgaatttgtg

SEQ ID NO: 40 - RNA, 22 nt, synthetic construct
  source 1..22: mol_type = other RNA
cactagattgtgagctgctgga

SEQ ID NO: 41 - RNA, 22 nt, synthetic construct
  source 1..22: mol_type = other RNA
tcagtgcactacagaactttgt

SEQ ID NO: 42 - RNA, 22 nt, synthetic construct
  source 1..22: mol_type = other RNA
taacactgtctggtaacgatgt

SEQ ID NO: 43 - RNA, 22 nt, synthetic construct
  source 1..22: mol_type = other RNA
tggaatgtaaggaagtgtgtgg

SEQ ID NO: 44 - RNA, 22 nt, synthetic construct
  source 1..22: mol_type = other RNA
cctgttctccattacttggctc

SEQ ID NO: 45 - RNA, 22 nt, synthetic construct
  source 1..22: mol_type = other RNA
tgaggtagtaagttgtattgtt

SEQ ID NO: 46 - RNA, 22 nt, synthetic construct
  source 1..22: mol_type = other RNA
gtgtgcggaaatgcttctgcta

SEQ ID NO: 47 - RNA, 21 nt, synthetic construct
  source 1..21: mol_type = other RNA
atgacctatgatttgacagac

SEQ ID NO: 48 - RNA, 22 nt, synthetic construct
  source 1..22: mol_type = other RNA
atcacacaaaggcaacttttgt

SEQ ID NO: 49 - RNA, 22 nt, synthetic construct
  source 1..22: mol_type = other RNA
tagcagcacgtaaatattggcg

SEQ ID NO: 50 - RNA, 22 nt, synthetic construct
  source 1..22: mol_type = other RNA
taacactgtctggtaaagatgg

SEQ ID NO: 51 - RNA, 21 nt, synthetic construct
  source 1..21: mol_type = other RNA
ggtgcagtgctgcatctctgg

SEQ ID NO: 52 - RNA, 22 nt, synthetic construct
  source 1..22: mol_type = other RNA
tgtaacagcaactccatgtgga

SEQ ID NO: 53 - RNA, 21 nt, synthetic construct
  source 1..21: mol_type = other RNA
aatggcgccactagggttgtg

SEQ ID NO: 54 - RNA, 22 nt, synthetic construct
  source 1..22: mol_type = other RNA
tgaggtagtaggttgtatggtt

SEQ ID NO: 55 - RNA, 22 nt, synthetic construct
  source 1..22: mol_type = other RNA
ctgcgcaagctactgccttgct

SEQ ID NO: 56 - RNA, 22 nt, synthetic construct
  source 1..22: mol_type = other RNA
caaattcgtatctaggggaata

SEQ ID NO: 57 - RNA, 24 nt, synthetic construct
  source 1..24: mol_type = other RNA
tccctgagaccctttaacctgtga

SEQ ID NO: 58 - RNA, 22 nt, synthetic construct
  source 1..22: mol_type = other RNA
taacagtctacagccatggtcg

SEQ ID NO: 59 - RNA, 22 nt, synthetic construct
  source 1..22: mol_type = other RNA
catcatcgtctcaaatgagtct

SEQ ID NO: 60 - RNA, 22 nt, synthetic construct
  source 1..22: mol_type = other RNA
taacactgtctggtaaagatgg

SEQ ID NO: 61 - RNA, 21 nt, synthetic construct
  source 1..21: mol_type = other RNA
tgagatgaagcactgtagctc

SEQ ID NO: 62 - RNA, 22 nt, synthetic construct
  source 1..22: mol_type = other RNA
ggtgcagtgctgcatctctggt

SEQ ID NO: 63 - RNA, 22 nt, synthetic construct
  source 1..22: mol_type = other RNA
ggattcctggaaatactgttct

SEQ ID NO: 64 - RNA, 22 nt, synthetic construct
  source 1..22: mol_type = other RNA
tcagtgcactacagaactttgt

SEQ ID NO: 65 - RNA, 21 nt, synthetic construct
  source 1..21: mol_type = other RNA
tcagtgcatgacagaacttgg

SEQ ID NO: 66 - RNA, 22 nt, synthetic construct
  source 1..22: mol_type = other RNA
tagcagcacataatggtttgtg

SEQ ID NO: 67 - RNA, 22 nt, synthetic construct
  source 1..22: mol_type = other RNA
tagcagcacatcatggtttaca

SEQ ID NO: 68 - RNA, 22 nt, synthetic construct
  source 1..22: mol_type = other RNA
accactgaccgttgactgtacc

SEQ ID NO: 69 - RNA, 22 nt, synthetic construct
  source 1..22: mol_type = other RNA
aaccatcgaccgttgagtggac

SEQ ID NO: 70 - RNA, 22 nt, synthetic construct
  source 1..22: mol_type = other RNA
tgggtctttgcgggcgagatga

SEQ ID NO: 71 - RNA, 22 nt, synthetic construct
  source 1..22: mol_type = other RNA
aactggccctcaaagtcccgct

SEQ ID NO: 72 - RNA, 22 nt, synthetic construct
  source 1..22: mol_type = other RNA
tgtaacagcaactccatgtgga

SEQ ID NO: 73 - RNA, 21 nt, synthetic construct
  source 1..21: mol_type = other RNA
tagcagcacagaaatattggc

SEQ ID NO: 74 - RNA, 22 nt, synthetic construct
  source 1..22: mol_type = other RNA
acagtagtctgcacattggtta

SEQ ID NO: 75 - RNA, 23 nt, synthetic construct
  source 1..23: mol_type = other RNA
cccagtgtttagactatctgttc

SEQ ID NO: 76 - RNA, 22 nt, synthetic construct
  source 1..22: mol_type = other RNA
taacactgtctggtaacgatgt

SEQ ID NO: 77 - RNA, 23 nt, synthetic construct
  source 1..23: mol_type = other RNA
acagcaggcacagacaggcagty

SEQ ID NO: 78 - RNA, 25 nt, synthetic construct
  source 1..25: mol_type = other RNA
tcaagtcactagtggttccgtttag

SEQ ID NO: 79 - RNA, 21 nt, synthetic construct
  source 1..21: mol_type = other RNA
atcacattgccagggatttcc

SEQ ID NO: 80 - RNA, 22 nt, synthetic construct
  source 1..22: mol_type = other RNA
agagcttagctgattggtgaac

SEQ ID NO: 81 - RNA, 22 nt, synthetic construct
  source 1..22: mol_type = other RNA
tagcaccatctgaaatcggtta

SEQ ID NO: 82 - RNA, 22 nt, synthetic construct
  source 1..22: mol_type = other RNA
tagcaccatttgaaatcggtta

SEQ ID NO: 83 - RNA, 22 nt, synthetic construct
  source 1..22: mol_type = other RNA
ctttcagtcagatgtttgctgc

SEQ ID NO: 84 - RNA, 22 nt, synthetic construct
  source 1..22: mol_type = other RNA
ctttcagtcggatgtttacagc

SEQ ID NO: 85 - RNA, 22 nt, synthetic construct
  source 1..22: mol_type = other RNA
tgtaaacatccttgactggaag

SEQ ID NO: 86 - RNA, 22 nt, synthetic construct
  source 1..22: mol_type = other RNA
tttttcattattgctcctgacc

SEQ ID NO: 87 - RNA, 22 nt, synthetic construct
  source 1..22: mol_type = other RNA
gctgactcctagtccagggctc

SEQ ID NO: 88 - RNA, 22 nt, synthetic construct
  source 1..22: mol_type = other RNA
ttatcagaatctccaggggtac

SEQ ID NO: 89 - RNA, 22 nt, synthetic construct
  source 1..22: mol_type = other RNA
taatgcccctaaaaatccttat

SEQ ID NO: 90 - RNA, 22 nt, synthetic construct
  source 1..22: mol_type = other RNA
ttgtacatggtaggctttcatt

SEQ ID NO: 91 - RNA, 22 nt, synthetic construct
  source 1..22: mol_type = other RNA
tgaggtagtaagttgtattgtt

SEQ ID NO: 92 - RNA, 10 nt, synthetic construct
  source 1..10: mol_type = other RNA
aaatgagttc

SEQ ID NO: 93 - RNA, 10 nt, synthetic construct
  source 1..10: mol_type = other RNA
cagaatattg

SEQ ID NO: 94 - RNA, 10 nt, synthetic construct
  source 1..10: mol_type = other RNA
tatgtgcttt

SEQ ID NO: 95 - RNA, 10 nt, synthetic construct
  source 1..10: mol_type = other RNA
gacgcgttta

SEQ ID NO: 96 - RNA, 22 nt, synthetic construct
  source 1..22: mol_type = other RNA
agagaacagaaaaccaagggaa

SEQ ID NO: 97 - RNA, 22 nt, synthetic construct
  source 1..22: mol_type = other RNA
tgagaactgaaatccatgggtt

SEQ ID NO: 98 - RNA, 22 nt, synthetic construct
  source 1..22: mol_type = other RNA
tgagaactgaataccatgggtt